(19) United States
(12) Patent Application Publication
(10) Pub. No.: US 2008/0050379 A1
(43) Pub. Date: Feb. 28, 2008

(54) Cancer gene determination and therapeutic screening using signature gene sets

(76) Inventors: Paul E. Young, Gaithersburg, MD (US); Meena Augustus, Burtonsville, MD (US); Kenneth C. Carter, Potomac, MD (US); Reinhard Ebner, Gaithersburg, MD (US); Gregory Endress, Florence, MA (US); Stephen K. Horrigan, Potomac, MD (US); Daniel R. Soppet, Centerville, VA (US); Zoe Weaver, Chevy Chase, MD (US)

Correspondence Address: Alan J. Grant, Esq.;c/o Carella, Byrne, Bain, Gilfillan, Cecchi,, Stewart & Olstein, 5 Becker Farm Road, Roseland, NJ, 07068, US

(21) Appl. No.: 11/881,252

(22) Filed: Jul. 26, 2007

Related U.S. Application Data
(63) Continuation of application No. 10/843,641, filed on May 11, 2004, now abandoned.
Application No. 10/843,641 is a continuation-in-part of application No. 09/873,367, filed on Jun. 5, 2001, now abandoned.
Application No. 10/843,641 is a continuation-in-part of application No. 09/954,531, filed on Sep. 18, 2001, now abandoned.
Application No. 10/843,641 is a continuation-in-part of application No. 09/962,436, filed on Sep. 25, 2001, now abandoned.
Application No. 10/843,641 is a continuation-in-part of application No. 09/954,456, filed on Sep. 18, 2001, now abandoned.
Application No. 10/843,641 is a continuation-in-part of application No. 09/962,832, filed on Sep. 25, 2001, now abandoned.
Application No. 10/843,641 is a continuation-in-part of application No. 09/964,824, filed on Sep. 27, 2001, now abandoned.
Application No. 10/843,641 is a continuation-in-part of application No. 09/967,768, filed on Sep. 28, 2001, now abandoned.
Application No. 10/843,641 is a continuation-in-part of application No. 09/968,007, filed on Oct. 2, 2001, now abandoned.
Application No. 10/843,641 is a continuation-in-part of application No. 09/969,347, filed on Oct. 2, 2001, now abandoned.
Application No. 10/843,641 is a continuation-in-part of application No. 09/969,708, filed on Oct. 3, 2001, now abandoned.
(60) Provisional application No. 60/209,473, filed on Jun. 5, 2000.
(60) Provisional application No. 60/209,531, filed on Jun. 5, 2000.
(60) Provisional application No. 60/236,842, filed on Sep. 29, 2000.
(60) Provisional application No. 60/236,891, filed on Sep. 29, 2000.
(60) Provisional application No. 60/244,867, filed on Nov. 1, 2000.
(60) Provisional application No. 60/245,084, filed on Nov. 1, 2000.
(60) Provisional application No. 60/233,133, filed on Sep. 18, 2000.
(60) Provisional application No. 60/234,009, filed on Sep. 20, 2000.
(60) Provisional application No. 60/234,034, filed on Sep. 20, 2000.
(60) Provisional application No. 60/234,509, filed on Sep. 22, 2000.
(60) Provisional application No. 60/234,567, filed on Sep. 22, 2000.
(60) Provisional application No. 60/235,082, filed on Sep. 25, 2000.
(60) Provisional application No. 60/234,924, filed on Sep. 25, 2000.
(60) Provisional application No. 60/233,617, filed on Sep. 18, 2000.
(60) Provisional application No. 60/234,052, filed on Sep. 20, 2000.
(60) Provisional application No. 60/234,923, filed on Sep. 25, 2000.
(60) Provisional application No. 60/235,134, filed on Sep. 25, 2000.
(60) Provisional application No. 60/235,637, filed on Sep. 26, 2000.
(60) Provisional application No. 60/235,638, filed on Sep. 26, 2000.
(60) Provisional application No. 60/235,711, filed on Sep. 27, 2000.
(60) Provisional application No. 60/235,720, filed on Sep. 27, 2000.
(60) Provisional application No. 60/235,840, filed on Sep. 27, 2000.
(60) Provisional application No. 60/235,863, filed on Sep. 27, 2000.
(60) Provisional application No. 60/235,077, filed on Sep. 25, 2000.
(60) Provisional application No. 60/235,280, filed on Sep. 25, 2000.
(60) Provisional application No. 60/236,033, filed on Sep. 28, 2000.
(60) Provisional application No. 60/236,032, filed on Sep. 28, 2000.
(60) Provisional application No. 60/236,028, filed on Sep. 28, 2000.
(60) Provisional application No. 60/236,109, filed on Sep. 28, 2000.
(60) Provisional application No. 60/236,034, filed on Sep. 28, 2000.
(60) Provisional application No. 60/236,111, filed on Sep. 28, 2000.
(60) Provisional application No. 60/237,172, filed on Oct. 2, 2000.
(60) Provisional application No. 60/237,173, filed on Oct. 2, 2000.
(60) Provisional application No. 60/237,278, filed on Oct. 2, 2000.
(60) Provisional application No. 60/237,294, filed on Oct. 2, 2000.
(60) Provisional application No. 60/237,295, filed on Oct. 2, 2000.
(60) Provisional application No. 60/237,316, filed on Oct. 2, 2000.
(60) Provisional application No. 60/237,598, filed on Oct. 3, 2000.
(60) Provisional application No. 60/237,604, filed on Oct. 3, 2000.
(60) Provisional application No. 60/237,606, filed on Oct. 3, 2000.
(60) Provisional application No. 60/237,608, filed on Oct. 3, 2000.
(60) Provisional application No. 60/237,425, filed on Oct. 3, 2000.

(51) Int. Cl. A61K 39/395; A61K 31/00; A61P 43/00; C07K 16/18; C12Q 1/68; C40B 30/06; G01N 33/566

(52) U.S. Cl. 424/138.1; 424/178.1; 435/6; 436/501; 506/10; 514/789; 530/387.1; 530/391.7

(57) ABSTRACT

Processes for assaying potential antitumor agents based on their modulation of the expression of specified genes, or sets, of suspected cancer cell genes are disclosed, along with methods for diagnosing cancerous, or potentially cancerous, conditions as a result of the expression, or patterns of expression, of such genes, or sets of genes. Also disclosed are methods for determining functionally related genes, or gene sets, as well as methods for treating cancer based on targeting expression products of such genes, or gene sets, and determining genes involved in the cancerous process.

This application is a Continuation-in-Part of U.S. application Ser. No. 09/873,367, filed 5 Jun. 2001, which claimed priority of U.S. Provisional Application 60/209,473, filed 5 Jun. 2000; 60/209,531, filed 5 Jun. 2000; 60/236,842, filed 29 Sep. 2000; 60/236,891, filed 29 Sep. 2000; 60/244,867, filed 1 Nov. 2000; and 60/245,084, filed 1 Nov. 2000,

and of U.S. application Ser. No. 09/954,531, filed 18 Sep. 2001, which claimed priority of 60/233,133, filed 18 Sep. 2000; 60/234,009, filed 20 Sep. 2000; 60/234,034, filed 20 Sep. 2000; 60/234,509, filed 22 Sep. 2000; 60/234,567, filed 22 Sep. 2000;

and of U.S. application Ser. No. 09/954,456, filed 18 Sep. 2001, which claimed priority of U.S. Provisional Application 60/233,617, filed 18 Sep. 2000; 60/234,052, filed 20 Sep. 2000; 60/234,923, filed 25 Sep. 2000; 60/235,134, filed 25 Sep. 2000; 60/235,637, filed 26 Sep. 2000; 60/235,638, filed 26 Sep. 2000; 60/235,711, filed 27 Sep. 2000; 60/235,720, filed 27 Sep. 2000; 60/235,840, filed 27 Sep. 2000; 60/235,863, filed 27 Sep. 2000;

and of U.S. application Ser. No. 09/962,436, filed 25 Sep. 2001, which claimed priority of U.S. Provisional Application 60/235,082, filed 25 Sep. 2000, and 60/234,924, filed 25 Sep. 2000;

and of U.S. application Ser. No. 09/962,832, filed 25 Sep. 2001, which claimed priority of U.S. Provisional Application 60/235,077, filed 25 Sep. 2000; 60/235,280, filed 25 Sep. 2000;

and of U.S. application Ser. No. 09/964,824, filed 27 Sep. 2001, which claimed priority of U.S. Provisional Application 60/236,028, filed 28 Sep. 2000; 60/236,032, filed 28 Sep. 2000; 60/236,033, filed 28 Sep. 2000;

and of U.S. application Ser. No. 09/967,768, filed 28 Sep. 2001, which claimed priority of U.S. Provisional Application 60/236,034, filed 28 Sep. 2000; 60/236,109, filed 28 Sep. 2000; 60/236,111, filed 28 Sep. 2000;

and of U.S. application Ser. No. 09/968,007, filed 2 Oct. 2001, which claimed priority of U.S. Provisional Application 60/237,172, filed 2 Oct. 2000; 60/237,173, filed 2 Oct. 2000; 60/237,278, filed 2 Oct. 2000; 60/237,294, filed 2 Oct. 2000; 60/237,295, filed 2 Oct. 2000; 60/237,316, filed 2 Oct. 2000;

and of U.S. application Ser. No. 09/969,347, filed 2 Oct. 2001, which claimed priority of U.S. Provisional Application 60/237,598, filed 3 Oct. 2000 and 60/237,604, filed 3 Oct. 2000,

and of U.S. application Ser. No. 09/969,708, filed 3 Oct. 2001, which claimed priority of U.S. Provisional Application 60/237,606, filed 3 Oct. 2000, 60/237,608, filed 3 Oct. 2000, and 60/237,425, filed 3 Oct. 2000

the disclosures of all of which, including all sequence listings and drawings contained therein, are hereby incorporated by reference in their entirety.

### FIELD OF THE INVENTION

The present invention relates to methods of assaying potential anti-tumor agents based on their modulation of the expression of specified sets of genes and methods for diagnosing cancerous, or potentially cancerous, conditions as a result of the patterns of expression of such gene sets.

### BACKGROUND OF THE INVENTION

Screening assays for novel drugs are based on the response of model cell based systems in vitro to treatment with specific compounds. Various measures of cellular response have been utilized, including the release of cytokines, alterations in cell surface markers, activation of specific enzymes, as well as alterations in ion flux and/or pH. Some such screens rely on specific genes, such as oncogenes (or gene mutations).

### BRIEF SUMMARY OF THE INVENTION

In accordance with the present invention, there are provided characteristic sets of gene sequences whose expression, or non-expression, or change in expression, either an increase or decrease thereof, are indicative of the cancerous or non-cancerous status of a given cell. More particularly, such genes whose expression is changed in cancerous, as compared to non-cancerous cells, from a specific tissue (in particular, any of those disclosed herein) are genes that include one of the nucleotide sequences of SEQ ID NO: 1-8447, or sequences that are substantially identical to said sequences.

Such a change in expression may be an increase or a decrease in expression or activity of the gene or gene sequences disclosed herein.

It is another object of the present invention to provide methods of using such characteristic, or signature, gene sets as a basis for assaying the potential ability of selected chemical agents to modulate upward or downward the expression of said characteristic, or signature, gene sets.

It is a further object of the present invention to provide methods of detecting the expression, or non-expression, or amount of expression, of said characteristic, or signature, gene sets, or portions thereof, as a means of determining the cancerous, or non-cancerous, status (or potential cancerous status) of selected cells as grown in culture or as maintained in situ.

It is a still further object of the present invention to provide methods for treating cancerous conditions utilizing selected chemical agents as determined from their ability to modulate (i.e., increase or decrease) the selected characteristic, or signature, gene sets as disclosed herein, where said genes include, or comprise, one of the sequences of SEQ ID NO: 1-8447, or sequences substantially identical to said sequences, or characteristic portions of said sequences.

In another aspect, the present invention relates to a process for identifying an agent that modulates the activity of a cancer-related gene comprising:

(a) contacting a compound with a cell containing a gene corresponding to (such as a gene that encodes an RNA at least 90% identical to the RNA encoded by or complementary to) a polynucleotide comprising, or having, a sequence selected from the group consisting of the gene sequences of SEQ ID NO: 1-8447 and thereby identifying said gene as being a cancer initiating or facilitating gene. Said genes may, for example, be oncogenes, cancer facilitating or promoting genes, or cancer suppressor genes. Said agents may increase or decrease gene expression.

The present invention also relates to a process for identifying an anti-neoplastic agent comprising contacting a cell exhibiting neoplastic activity with a compound first identified as a cancer-related gene modulator using an assay process as disclosed herein for determining gene modulating activity and detecting a decrease in said neoplastic activity after said contacting compared to when said contacting does not occur.

In a further aspect, the present invention relates to a process for identifying an anti-neoplastic or anti-tumor agent comprising administering to an animal exhibiting a cancerous condition an effective amount of an agent first identified according to a process as disclosed herein and detecting a decrease in said cancerous condition thereby identifying such an agent, said decrease including the death of the cancerous cell or cells.

The present invention also relates to a process for determining the cancerous status of a cell, comprising determining the level of expression in said cell of at least one gene that corresponds to a polynucleotide comprising, or having, a sequence selected from the group consisting of SEQ ID NO: 1-8447 wherein an elevated expression relative to a known non-cancerous cell or a reduced expression relative to a known cancerous cell indicates a cancerous state or potentially cancerous state. Such sequence identity may include 100 percent identical as defined herein and any number of such genes may be used.

In an additional aspect, the present invention relates to a process for determining a cancer initiating or facilitating gene comprising contacting a cell expressing a test gene (i.e., a gene whose status as a cancer initiating or facilitating gene is to be determined) with an agent that decreases the expression of a gene that encodes an RNA at least 90%, preferably 95%, identical to an RNA encoded by (i.e., a gene corresponding to) a polynucleotide comprising, or having, a sequence selected from the group consisting of SEQ ID NO: 1-8447 and detecting a decrease in expression of said test gene compared to when said agent is not present, thereby identifying said test gene as being a cancer initiating or facilitating gene. Such genes may, of course, be oncogenes and said decrease in expression may be due to a decrease in copy number of said gene in said cell or a cell derived from said cell, such as where copy number is reduced in the cells formed by replication of such cells.

The present invention also relates to a process for determining a cancer suppressor gene comprising contacting a cell expressing a test gene (i.e., a gene whose status as a cancer suppressor gene is to be determined) with an agent that increases the expression of a gene that corresponds to (i.e., encodes an RNA at least 90%, preferably 95%, identical to an RNA encoded by or complementary to) a polynucleotide comprising a sequence selected from the group consisting of SEQ ID NO: 1-8447 and detecting an increase in expression of said test gene compared to when said agent is not present, thereby identifying said test gene as being a cancer suppressor gene. The sequence identity may include identical sequences, as defined herein, and such a process includes embodiments wherein the increase in expression is due to an increase in copy number of the gene in said cell or a cell derived from said cell, such as following cellular replication.

In another aspect, the present invention relates to a process for treating cancer comprising contacting a cancerous cell with an agent having activity against an expression product encoded by a gene corresponding to a polynucleotide comprising a nucleotide sequence selected from at least one of SEQ ID NO: 1-8447. Such a process includes an embodiment wherein the cancerous cell is contacted in vivo. The agent may include an antibody.

The present invention also relates to a method for producing a product comprising identifying an agent according to the assay processes of the invention wherein said product is the data collected with respect to said agent as a result of said process and wherein said data is sufficient to convey the chemical structure and/or properties of said agent.

The present invention further relates to a process for treating a cancerous condition in an animal afflicted therewith comprising administering to said animal a therapeutically effective amount of an agent first identified as having anti-neoplastic activity using one or more of the processes of the invention.

In a further aspect, the present invention relates to a process for protecting an animal against cancer comprising administering to an animal at risk of developing cancer a therapeutically effective amount of an agent first identified as having anti-neoplastic activity using one or more of the processes disclosed herein.

### Sequence Listing on CD-ROM Only

The sequences disclosed herein as SEQ ID NO: 1-8447 in the sequence listing are contained on compact disc (CD-ROM) only, which accompanies this application and the contents of said CD-ROMs are hereby incorporated by reference in their entirety.

### DETAILED SUMMARY OF THE INVENTION

The present invention relates to methods of assaying for potential antitumor agents based on their modulation of the expression of specified sets of genes and methods for diagnosing cancerous, or potentially cancerous, conditions as a result of the patterns of expression of such gene sets and for determining cancer-inducing or regulating genes, and gene sets, based on common expression or regulation of such genes, or gene sets.

In accordance with the present invention, model cellular systems using cell lines, primary cells, or tissue samples are maintained in growth medium and may be treated with compounds that may be at a single concentration or at a range of concentrations. At specific times after treatment, cellular RNAs are isolated from the treated cells, primary cells or tumors, which RNAs are indicative of expression of selected genes. The cellular RNA is then divided and subjected to analysis that detects the presence and/or quantity of specific RNA transcripts, which transcripts may then be amplified for detection purposes using standard methodologies, such as, for example, reverse transcriptase polymerase chain reaction (RT-PCR), etc. The presence or absence, or levels, of specific RNA transcripts are determined from these measurements and a metric derived for the type and degree of response of the sample to the treated compound compared to control samples.

In any of the methods of the invention, the cancer is one or more of colon cancer, lung cancer, ovarian cancer, pancreatic cancer, thyroid cancer, stomach cancer, prostate cancer, kidney cancer, esophageal cancer and/or breast cancer.

Also in accordance with the present invention, there are disclosed herein characteristic, or signature, sets of genes and gene sequences whose expression is, or can be, as a result of the methods of the present invention, linked to, or used to characterize, the cancerous, or non-cancerous, status of the cells, or tissues, to be tested. Thus, the methods of the present invention identify novel anti-neoplastic agents based on their alteration of expression of small sets of characteristic, or indicator, or signature genes in specific model systems. The methods of the invention may therefore be used with a variety of cell lines or with primary samples from tumors maintained in vitro under suitable culture conditions for varying periods of time, or in situ in suitable animal models.

More particularly, certain genes have been identified that are expressed at levels in cancer cells that are different than the expression levels in non-cancer cells. In one instance, the identified genes are expressed at higher levels in cancer cells than in normal cells. In another instance, the identified genes are expressed at lower levels in cancer cells as compared to normal cells.

In accordance with the foregoing, the present invention relates to a process for determining the cancerous status of a cell, comprising determining the level of expression in said cell of at least one gene that corresponds to (i.e., encodes an RNA at least 95% identical to the RNA encoded by or complementary to) a polynucleotide comprising a sequence selected from the group consisting of SEQ ID NO: 1-8447 wherein an elevated expression relative to a known non-cancerous cell or a reduced expression relative to a known non-cancerous cell indicates a cancerous state or potentially cancerous state. Such sequence identity may include 100 percent identical as defined herein and any number of such genes may be used.

Thus, the present invention also relates to a process for identifying an anti-neoplastic agent comprising contacting a cell exhibiting neoplastic activity with a compound first identified as a cancer-related gene modulator using an assay process as disclosed herein for determining gene modulating activity and detecting a decrease in said neoplastic activity after said contacting compared to when said contacting does not occur (i.e., comparing expression when said agent is present versus when said agent is not present).

In preferred embodiments of the present invention, such cancer is pancreatic cancer, such as a carcinoma, preferably adenocarcinoma.

In a further aspect, the present invention relates to a process for identifying an anti-neoplastic agent comprising administering to an animal exhibiting a cancerous condition an effective amount of an agent first identified as having such activity using a process as disclosed herein and detecting a decrease in said cancerous condition thereby identifying such an agent.

It should be kept in mind that the anti-tumor or anti-neoplastic agents identified by the processes of the invention include both novel agents whose structure and anti-tumor activity were not previously known prior to identification of their activity by the processes herein as well as non-novel agents, whose structure was known but whose therapeutic value as anti-tumor agents was not appreciated prior to identification by the assay processes of the invention.

In accordance with the foregoing, the present invention relates to a process for screening for an anti-neoplastic agent comprising the steps of:

(a) contacting a compound with a cell containing a polynucleotide comprising a nucleotide sequence selected from the group consisting of SEQ ID NO: 1-8447, or a sequence at least 90%, preferably at least 95%, identical thereto, under conditions wherein said polynucleotide is being expressed, and

(b) determining a change in expression of at least one of said polynucleotides,

wherein a change in expression is indicative of anti-neoplastic activity.

In particular embodiments, such change in expression may be an increase or a decrease in expression or activity. Of course, decreased expression of cancer initiating or facilitating genes is highly desirable, as is an increased expression of cancer suppressor genes.

More particularly, the present invention relates to a process for screening for an anti-neoplastic agent comprising the steps of:

(a) exposing a known cancerous cell to a chemical agent to be tested for antineoplastic activity;

(b) allowing said chemical agent to modulate the activity of one or more genes present in said cell wherein said genes include or comprise one of the sequences selected from the group consisting of the sequences of SEQ ID NO: 1-8447, sequences substantially identical to said sequences, or the complements of any of the foregoing;

(c) determining the expression of one or more genes of step (b);

(d) comparing the expression of said genes in the presence or absence of exposure to said chemical agent;

wherein a difference in expression is indicative of the ability of anti-neoplastic activity.

Thus, in one aspect, the present invention relates to a process for identifying an agent, such as an organic compound, that modulates the activity of a cancer-related gene, comprising:

(a) contacting a compound with a cell containing a gene that encodes an RNA at least 90%, preferably at least 95%, identical to the RNA encoded by (i.e., a gene that corresponds to) a polynucleotide comprising, or having, a sequence selected from the group consisting of SEQ ID NO: 1-8447 and under conditions promoting the expression of said gene; and

(b) detecting a difference in expression of said gene relative to when said compound is not present

thereby identifying an agent that modulates the activity of a cancer-related gene.

Such sequence identity includes embodiments wherein the RNAs are at least 97 or 98% identical in sequence as well as cases where the sequence is the same, thus where a gene encodes an RNA with the same nucleotide sequence as an RNA encoded by one of the sequences of SEQ ID NO: 1-8447.

In one embodiment of such processes, the sequence is selected from SEQ ID NO: 1-8447, and said difference in expression when said agent is present is a decrease in expression. Here, the gene used encodes an RNA like that encoded by (or at least 90% identical to) one of the sequences found to be expressed at an elevated level in cancer cells. In another such embodiment, the sequence is selected from SEQ ID NO: 1-8447 and said difference in expression is an increase in expression. The latter sequences encode RNAs found to be expressed at higher levels in normal cells, as opposed to cancer cells.

In specific embodiments of the present invention, said chemical agent to be tested modulates the expression of more than one said gene, especially where it modulates at least two said genes, more especially where at least 3, or at least 5 of said genes, or even 10 or more of said genes in said signature set, are modulated. In a preferred embodiment, this may include more than 10 (such as 20, 50 or even 100) or even all of said genes are modulated.

In one embodiment of the present invention, said gene modulation is downward modulation, so that, as a result of exposure to the chemical agent to be tested, one or more genes of the cancerous cell will be expressed at a lower level (or not expressed at all) when exposed to (i.e., contacted with) the agent as compared to the expression when not exposed to the agent (i.e., when said agent is not present).

In a preferred embodiment a selected set of said genes are expressed in the reference cell but not expressed in the cell to be tested as a result of the contacting or exposure of the test cell with the chemical agent. Thus, where said chemical agent causes the gene, or genes, of the tested cell to be expressed at a lower level than the same genes of the reference cell, this is indicative of downward modulation and indicates that the chemical agent to be tested has anti-neoplastic activity (or activity in reducing expression of such cancer-related genes).

In a separate embodiment, exposure of said cells to be tested to the chemical agent, especially one suspected of having anti-neoplastic activity, may result in upward modulation of said genes of the cell to be tested. Such upward modulation is interpreted as meaning that said genes are expressed where previously not expressed, or else are expressed in greater quantities, or at higher levels, when exposed to the agent as compared to non-exposure to the agent. Such upward modulation may be taken as indicative of anti-neoplastic activity by the tested chemical agent(s) of the gene, or genes, so modulated, resulting in lower neoplastic activity on the part of such cells, such as where increased expression of the gene, or genes, results in decreased growth and/or increased differentiation of said cells away from the cancerous state.

The genes useful in the assay processes include as a part thereof at least one of the sequences selected from the group consisting of the sequences of SEQ ID NO: 1-8447, or sequences substantially identical thereto. Such sequences also include sequences complementary to any of the sequences disclosed herein.

The genes identified by the present disclosure are considered “cancer-related” genes, as this term is used herein, and include genes expressed at higher levels (due, for example, to elevated rates of expression, elevated extent of expression or increased copy number) in cancer cells relative to expression of these genes in normal (i.e., non-cancerous) cells where said cancerous state or status of test cells or tissues has been determined by methods known in the art, such as by reverse transcriptase polymerase chain reaction (RT-PCR) as described in the Example below. In specific embodiments, this relates to the genes whose sequences correspond to the sequences of SEQ ID NO: 1-8447. Also specifically contemplated are genes whose expression is higher in normal as opposed to known cancer cells (as determined by other means, such as uncontrolled growth, change in antigenic surface proteins, genetic mutation, and the like) such that the decreased expression in cancer cells may be indicative of, or contributory to, the realization of the cancerous state. In specific embodiments thereof, this relates to the genes whose sequences correspond to the sequences of SEQ ID NO: 1-8447 disclosed herein. As used herein, the term “correspond” means that the gene has the indicated nucleotide sequence or that it encodes substantially the same RNA as would be encoded by the indicated sequence, the term “substantially” meaning about at least 90% identical as defined elsewhere herein and includes splice variants thereof.

The sequences disclosed herein may be genomic in nature and thus represent the sequence of an actual gene, such as a human gene, or may be a cDNA sequence derived from a messenger RNA (mRNA) and thus represent contiguous exonic sequences derived from a corresponding genomic sequence or they may be wholly synthetic in origin for purposes of detecting. As described in the Example, the expression of these cancer-related genes is determined from the relative expression levels of the RNA complement of a cancerous cell relative to a normal (i.e., non-cancerous) cell. Because of the processing that may take place in transforming the initial RNA transcript into the final mRNA, the sequences disclosed herein may represent less than the full genomic sequence. They may also represent sequences derived from ribosomal and transfer RNAs. Consequently, the genes present in the cell (and representing the genomic sequences) and the sequences disclosed herein, which are mostly cDNA sequences, may be identical or may be such that the cDNAs contain less than the full genomic sequence. Such genes and cDNA sequences are still considered corresponding sequences because they both encode similar RNA sequences. Thus, by way of non-limiting example only, a gene that encodes an RNA transcript, which is then processed into a shorter mRNA, is deemed to encode both such RNAs and therefore encodes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous or normal cells used to determine relative levels of expression because they represent the same sequences or are complementary to RNAs encoded by these genes. Such genes also include different alleles and splice variants that may occur in the cells used in the processes of the invention.

The genes of the invention “correspond to” a polynucleotide having a sequence of SEQ ID NO: 1-8447 if the gene encodes an RNA (processed or unprocessed, including naturally occurring splice variants and alleles) that is at least 90% identical, preferably at least 95% identical, most preferably at least 98% identical to, and especially identical to, an RNA that would be encoded by, or be complementary to, such as by hybridization with, a polynucleotide having the indicated sequence. In addition, genes including sequences at least 90% identical to a sequence selected from SEQ ID NO: 1-8447, preferably at least about 95% identical to such a sequence, more preferably at least about 98% identical to such sequence and most preferably comprising such sequence are specifically contemplated by all of the processes of the present invention as being genes that correspond to these sequences. In addition, sequences encoding the same proteins as any of these sequences, regardless of the percent identity of such sequences, are also specifically contemplated by any of the methods of the present invention that rely on any or all of said sequences, regardless of how they are otherwise described or limited. Thus, any such sequences are available for use in carrying out any of the methods disclosed according to the invention. Such sequences also include any open reading frames, as defined herein, present within any of the sequences of SEQ ID NO: 1-8447.

Further in accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100 [1-(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to a polynucleotides, such terms refer to the products produced by treatment of said polynucleotides with any of the common endonucleases, or any stretch of polynucleotides that could be synthetically synthesized.

As used herein and except as noted otherwise, all terms are defined as given below.

In accordance with the present invention, the term “DNA segment” or “DNA sequence” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, using a cloning vector. Such segments are provided in the form of an open reading frame uninterrupted by internal nontranslated sequences, or introns, which are typically present in eukaryotic genes. Sequences of non-translated DNA may be present downstream from the open reading frame, where the same do not interfere with manipulation or expression of the coding regions.

The term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

In accordance with the present invention, the term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s).

The term “fragment,” when referring to a coding sequence, means a portion of DNA comprising less than the complete coding region whose expression product retains essentially the same biological function or activity as the expression product of the complete coding region.

The term “primer” means a short nucleic acid sequence that is paired with one strand of DNA and provides a free 3′-OH end at which a DNA polymerase starts synthesis of a deoxyribonucleotide chain.

The term “promoter” means a region of DNA involved in binding of RNA polymerase to initiate transcription. The term “enhancer” refers to a region of DNA that, when present and active, has the effect of increasing expression of a different DNA sequence that is being expressed, thereby increasing the amount of expression product formed from said different DNA sequence.

The term “open reading frame (ORF)” means a series of triplets coding for amino acids without any termination codons and is a sequence (potentially) translatable into protein.

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

In carrying out the assays of the invention, relative antineoplastic activity may be ascertained by the extent to which a given chemical agent modulates the expression of genes present in a cancerous cell. Thus, a first chemical agent that modulates the expression of a gene associated with the cancerous state (i.e., a gene that includes one of the sequences disclosed herein and present in cancerous cells) to a larger degree than a second chemical agent tested by the assays of the invention is thereby deemed to have higher, or more desirable, or more advantageous, anti-neoplastic activity than said second chemical agent. Alternatively, where first and second chemical agents modulate expression of more than one of said genes, but where the second modulates expression of, for example, five said genes, whereas the first modulates expression of only three of said genes, especially where the three form a subset of the five, then the second chemical agent is deemed a more potent anti-neoplastic agent than the first. Such anti-neoplastic activity, as determined using the assays of the present invention, may necessarily include combinations of the foregoing possibilities, which are in no way to be considered limiting.

In utilizing these gene sequences for the assays according to the invention, the genes whose activity is to be determined with and without the presence of the compound to be evaluated for anti-tumor activity may be any one, or several, or any combination of the gene sequences disclosed herein as SEQ ID NO: 1-8447. However, how the gene sequences are employed in such assays depends on the pattern of gene expression disclosed for the signature sets. For example, a sequence that is expressed in cancerous cells but not in normal cells will identify a potential anticancer agent by that agent's ability to decrease expression of the sequence, or sequences, in tumor cells. Conversely, a sequence, or sequences, expressed in normal but not tumor cells will identify a potential antitumor agent by its ability to increase expression of those genes in the tumor cells. The same relationship holds true where the sequences are expressed in both cancer and normal cells but are expressed at a higher level in one than in the other, and vice versa. Based on the expression patterns disclosed for the gene sequences and signature sets disclosed herein, it should be readily apparent to those skilled in the art how to conduct assays for potential antitumor agents using the signature gene sets. The same holds true where the sequences, or signature gene sets, are utilized to determine the cancerous state of a cell or use of an agent to treat a cancerous condition.

Thus, in one aspect, the present invention relates to a process for screening for an anti-neoplastic agent comprising the steps of (a) exposing cells to a chemical agent to be tested for antineoplastic activity, and (b) determining a change in expression of at least one gene of a signature gene set, or a sequence that is at least 90%, preferably at least 95% identical thereto, wherein a change in expression is indicative of anti-neoplastic activity. Such change in expression is intended to mean a change that includes any activity of the gene, and may be an increase or decrease thereof. In addition, such change in activity may be a change in expression or other activity of at least 1 such gene, such as 5 or 10, or more of the genes of a signature set, even as many as half of such genes or even of all of the genes of a particular gene set.

The gene expression to be measured is commonly assayed using RNA expression as an indicator. Thus, the greater the level of RNA (such as a messenger RNA) detected the higher the level of expression of the corresponding gene. Thus, gene expression, either absolute or relative, such as where the expression of several different genes are being quantitatively evaluated and compared, for example, where chemical agents modulate the expression of more than one gene, such as a set of 3, 4, 5, or more genes, is determined by the relative expression of the RNAs encoded by such genes.

RNA may be isolated from samples in a variety of ways, including lysis and denaturation with a phenolic solution containing a chaotropic agent (e.g., triazol) followed by isopropanol precipitation, ethanol wash, and resuspension in aqueous solution; or lysis and denaturation followed by isolation on solid support, such as a Qiagen resin and reconstitution in aqueous solution; or lysis and denaturation in non-phenolic, aqueous solutions followed by enzymatic conversion of RNA to DNA template copies.

Normally, prior to applying the processes of the invention, steady state RNA expression levels for the genes, and sets of genes, disclosed herein will have been obtained. It is the steady state level of such expression that is affected by potential anti-neoplastic agents as determined herein. Such steady state levels of expression are easily determined by any methods that are sensitive, specific and accurate. Such methods include, but are in no way limited to, real time quantitative polymerase chain reaction (PCR), for example, using a Perkin-Elmer 7700 sequence detection system with gene specific primer probe combinations as designed using any of several commercially available software packages, such as Primer Express software, solid support based hybridization array technology using appropriate internal controls for quantitation, including filter, bead, or microchip based arrays, solid support based hybridization arrays using, for example, chemiluminescent, fluorescent, or electrochemical reaction based detection systems.

In one embodiment of the present invention, a set of genes useful in evaluating, or screening, or otherwise assaying, one or more chemical agents for anti-neoplastic activity in the assays disclosed herein will have already been shown to have differences in the ratios of steady state RNA levels in cancer cells, or tissues, relative to normal, or non-tumorous cells or tissues, or will have exhibited differences in the expression ratios in tumor samples compared to normal samples between genes in a given subset of the set of genes disclosed herein, or will have gene expression that has increased from undetectable levels to detectable levels, or vice versa, as the case may be, especially where sensitive detection methods are employed, or conversely will have decreased from detectable levels to undetectable levels with such procedures, especially sensitive procedures.

The genes, and gene sequences, useful in practicing the methods of the present invention are genes that are found to be selectively expressed in, or not expressed in, cancer cells as compared to non-cancer cells, or in which expression is down-regulated or up-regulated, as the case may be, in cancerous cells as compared to non-cancerous cells. Thus, these may include genes, or sets of genes, expressed in cancer cells but absent from, or inactive in, non-cancerous cells, or may include genes, or sets of genes, expressed in non-cancerous cells, but not expressed in cancer cells. Alternatively, the genes useful in practicing the present invention may be more expressed, or less expressed, in a cancerous cell relative to a non-cancerous cell. Such genes are generally those comprising the sequences of SEQ ID NO: 1-8447, with some exhibiting elevated expression in a cancerous versus non-cancerous cell and other exhibiting elevated expression in a non-cancerous versus a cancerous cell.

In accordance with the foregoing, the present invention further relates to a process for determining the cancerous status of a test cell, comprising determining expression in said test cell of at least one gene that corresponds to, or includes, one of the nucleotide sequences selected from the sequences of SEQ ID NO: 1-8447, or a nucleotide sequence that is at least 90%, preferably at least 95%, identical thereto, and then comparing said expression to expression of said at least one gene in at least one cell known to be non-cancerous whereby a difference in said expression indicates that said cell is cancerous.

In a particular embodiment, the present invention is directed to a process for determining the cancerous status of a cell to be tested, comprising determining the presence in said cell of at least one gene that includes one of the nucleotide sequences selected from the sequences of SEQ ID NO: 1-8447, including sequences having substantial identity homologous to said sequences, or characteristic fragments thereof, or the complements of any of the foregoing and then comparing the pattern of said gene presence and/or absence with that found for a cell known, or believed, to be non-cancerous, or normal, at least with respect to its genetic complement.

With respect to genes that correspond to at least one of the sequences of SEQ ID NO: 1-8447, up regulation of expression in cancer cells (as compared to non-cancer cells, which may lack said genes, or said gene expression, altogether) is indicative of a cancerous, or potentially cancerous, condition.

In specific embodiments, the present invention relates to embodiments wherein the genetic pattern is the modulation of expression of more than one gene, preferably 3, 4, or 5 genes, and even includes patterns where there is a modulation of expression of as many as 10, or more, genes. Thus, where a genetic pattern is the modulation of expression of 5 genes in a cancerous cell as compared to a non-cancerous cell from the same tissue type, such as a cancerous cell, versus a non-cancerous colon cell, such a pattern indicates a likelihood that such genes (i.e., the modulation of expression of those 5 genes) is an indicator of cancerous status and thereby provides a means of diagnosing a cancerous, or potentially cancerous, status. The absence of a specific set of genes from cancerous cells where said genes are present in otherwise normal cells, especially those of a similar type, is also indicative of a correlation with the cancerous state and thus can likewise be used as a means of diagnosing the cancerous state in other cells suspected of being cancerous.

For example, by way of non-limiting example, with respect to colon, especially colon adenocarcinoma, this would include SEQ ID NO: 1-333 (expressed in normal colon cells but not in colon cancer cells), SEQ ID NO: 334-522 (expressed at elevated levels in colon adenocarcinoma but not expressed in normal colon cells), SEQ ID NO: 523-837 (expressed at reduced levels, more than 2.09 fold, in colon adenocarcinoma but not in normal colon cells) and SEQ ID NO: 838-1067 (expressed at elevated (at least 2.1 fold) levels in colon adenocarcinoma but not elevated in normal colon cells). Thus, for colon the above groupings of sequences represent four signature gene sets for colon. For example, SEQ ID NO: 334-522 would represent a signature set or signature gene set for colon. The same is true for each of the organs and tissues listed below with their respective signature sets or signature gene sets.

In the same way as for colon, other gene sequences are indicative of the cancerous or normal state of other organs and tissues. Thus, as disclosed herein, these would include SEQ ID NO: 1068-2459 for breast, wherein SEQ ID NO: 1068-1255 represent genes expressed in infiltrating ductal carcinoma of the breast that are not expressed at detectable levels in normal breast, wherein SEQ ID NO: 1256-1459 represent genes expressed in breast carcinoma that are not expressed at detectable levels in normal breast, wherein SEQ ID NO: 1459-1664 represent genes expressed in infiltrating lobular carcinoma of the breast that are not expressed at detectable levels in normal breast, wherein SEQ ID NO: 1665-2067 represent genes expressed in normal breast that are absent or not expressed in infiltrating ductal carcinoma of the breast, and wherein SEQ ID NO: 2068-2459 represent genes expressed in normal breast cells but absent or not expressed in infiltrating lobular carcinoma of the breast that are not expressed at detectable levels in normal breast.

This further includes SEQ ID NO: 2460-3027 for stomach, wherein SEQ ID NO: 2460-2773 represent genes or gene sequences expressed in stomach cancer that are not expressed at detectable levels in normal stomach cells, and wherein SEQ ID NO: 2774-3027 represent genes or gene sequences expressed in normal stomach cells cancer that are not expressed at detectable levels in stomach cancer cells.

This further includes SEQ ID NO:3028-5303 for lung, wherein SEQ ID NO: 3028-3119 represent genes or gene sequences expressed in lung adenocarcinoma that are not expressed at appreciable levels in normal lung cells, wherein SEQ ID NO: 3120-3322 represent genes or gene sequences expressed in normal lung cells that are not expressed at appreciable levels in lung adenocarcinoma, wherein SEQ ID NO: 3323-3570 represent genes or gene sequences expressed in non-cancerous lung tissue that are not expressed at appreciable levels in malignant lung samples, wherein SEQ ID NO: 3571-3777 represent genes or gene sequences expressed in malignant lung samples that are not expressed at appreciable levels in non-malignant lung cells, wherein SEQ ID NO: 3778-3836 represent genes or gene sequences expressed in both normal and malignant lung adenocarcinoma but are up-regulated by at least about 2 fold in lung adenocarcinoma, wherein SEQ ID NO: 3837-3980 represent genes or gene sequences expressed at appreciable levels in normal lung samples but are not typically expressed in lung squamous cell carcinoma, wherein SEQ ID NO: 3981-4215 represent genes or gene sequences expressed in normal lung tissue but not ordinarily expressed in neuroendocrine carcinoma of the lung, wherein SEQ ID NO: 4216-4634 represent genes or gene sequences expressed at appreciable levels in lung neuroendocrine carcinoma that are not expressed at detectable levels in normal lung, wherein SEQ ID NO: 4635-4877 represent genes or gene sequences expressed in lung squamous cell carcinoma that are not expressed at detectable levels in normal lung, and wherein SEQ ID NO: 4878-5303 represent genes or gene sequences expressed in normal lung and lung adenocarcinoma but are down-regulated or under-expressed in lung adenocarcinoma relative to normal lung tissues.

This further includes SEQ ID NO: 5304-5886 for thyroid, wherein SEQ ID NO: 5304-5408 represent genes or gene sequences expressed in thyroid papillary carcinoma that are not found in normal thyroid tissue, wherein SEQ ID NO: 5409-5602 represent genes or gene sequences expressed in normal thyroid cells that are not expressed in thyroid papillary carcinoma and wherein SEQ ID NO: 5603-5886 represent genes or gene sequences expressed at a level at least about a 5 fold higher level in thyroid papillary carcinoma relative to normal thyroid cells.

This further includes SEQ ID NO: 5887-6147 for esophagus, wherein SEQ ID NO: 5887-6015 represent genes or gene sequences expressed in esophagus adenocarcinoma but not in normal esophagus from the same patients and wherein SEQ ID NO: 6016-6147 represent genes or gene sequences expressed in normal esophagus but not in esophagus adenocarcinoma samples from the same patients.

This further includes SEQ ID NO: 6148-6472 for ovary, wherein SEQ ID NO: 6148-6371 represent genes or gene sequences expressed only in malignant ovarian carcinomas, wherein SEQ ID NO: 6372-6424 represent genes or gene sequences expressed only in normal ovarian tissues and wherein SEQ ID NO: 6425-6472 represent genes or gene sequences expressed only in metastatic ovarian cancer.

This further includes SEQ ID NO: 6473-7473 for kidney, wherein SEQ ID NO:6473-6615 represent genes or gene sequences expressed in normal kidney but not in clear cell carcinoma of the kidney, wherein SEQ ID NO: 6616-6685 represent genes or gene sequences expressed in clear cell carcinoma cells but not in normal kidney cells, wherein SEQ ID NO: 6686-6973 represent genes or gene sequences expressed in normal kidney cells but not in renal cell carcinoma of the kidney, wherein SEQ ID NO: 6974-7156 represent genes or gene sequences expressed in renal cell carcinoma but not in normal kidney, wherein SEQ ID NO: 7157-7229 represent genes or gene sequences expressed in normal kidney but not in Wilm's tumor cells, and wherein SEQ ID NO: 7230-7473 represent genes or gene sequences expressed in Wilm's tumor but not in normal kidney cells.

This further includes SEQ ID NO: 7474-8131 for prostate, wherein SEQ ID NO: 7475-7833 represent genes or gene sequences expressed in prostate adenocarcinoma but not appreciably expressed in normal prostate cells, wherein SEQ ID NO: 7834-8071 represent genes or gene sequences expressed in normal prostate cells but not expressed at appreciable levels in prostate adenocarcinoma and wherein SEQ ID NO: 8072-8131 represent genes or gene sequences for ribosomal proteins that are highly expressed in prostate adenocarcinoma but are not expressed at appreciable levels in normal prostate cells.

This further includes SEQ ID NO: 8132-8447 for pancreas, wherein SEQ ID NO: 8132-8358 represent genes or gene sequences expressed in normal pancreas but not in pancreas adenocarcinoma and wherein SEQ ID NO: 8359-8447 represent genes or gene sequences expressed in pancreas adenocarcinoma but not in normal pancreas.

The gene patterns indicative of a cancerous state need not be characteristic of every cell found to be cancerous. Thus, the methods disclosed herein are useful for detecting the presence of a cancerous condition within a tissue where less than all cells exhibit the complete pattern. For example, a set of selected genes, comprising sequences homologous under stringent conditions (i.e., at least 95% identical) to at least one of the sequences of SEQ ID NO: 1-8447 and wherein the signature set is comprised of genes expressed and/or up-regulated in cancer cells relative to normal cells, as disclosed above for the signature gene set (or sets) used for practicing the invention, may be found, using appropriate probes, either DNA or RNA, to be present in as little as 60% of cells derived from a sample of tumorous, or malignant, tissue while being absent from as much as 60% of cells derived from corresponding non-cancerous, or otherwise normal, tissue (and thus being present in as much as 40% of such normal tissue cells). In a preferred embodiment, such gene pattern is found to be present in at least 70% of cells drawn from a cancerous tissue and absent from at least 70% of a corresponding normal, non-cancerous, tissue sample. In an especially preferred embodiment, such gene pattern is found to be present in at least 80% of cells drawn from a cancerous tissue and absent from at least 80% of a corresponding normal, non-cancerous, tissue sample. In a most preferred embodiment, such gene pattern is found to be present in at least 90% of cells drawn from a cancerous tissue and absent from at least 90% of a corresponding normal, non-cancerous, tissue sample. In an additional embodiment, such gene pattern is found to be present in at least 100% of cells drawn from a cancerous tissue and absent from at least 100% of a corresponding normal, non-cancerous, tissue sample, although the latter embodiment may represent a rare occurrence.

Conversely, where the signature set (including sequences from SEQ ID NO: 1-8447) is expressed or up-regulated in normal cells versus cancerous cells, as disclosed herein, expression in the normal cells but not in suspected cancerous cells may confirm a cancerous state in a suspected cancerous sample where the cells would show lower than expected expression of genes corresponding to one of these sequences.

Although the presence or absence of expression of one or more selected gene sequences may be indicative of a cancerous status for a given cell, the mere presence or absence of such a gene pattern may not alone be sufficient to achieve a malignant condition and thus the level of expression of such gene pattern may also be a significant factor in determining the attainment of a cancerous state. While a pattern of gene expression may be present in both cancerous and non-cancerous cells, the relative level of expression, as determined by any of the methods disclosed herein, all of which are well known in the art, may differ between the cancerous versus the non-cancerous cells. Thus, it becomes essential to also determine the level of expression of one or more of said genes as a separate means of diagnosing the presence of a cancerous status for a given cell, groups of cells, or tissues, either in culture or in situ.

In accordance with the invention disclosed herein, a determination of an anticancer agent using the signature gene sets described herein is based on patterns of modulation of such genes so that increase or decrease in expression of a gene due to the presence of such a potential agent may or may not be meaningful. Thus, the more genes in a gene set as disclosed herein that are affected by said agent the more likely said agent is an effective therapeutic agent.

In addition, different agents may have different abilities to affect the genes of a signature gene set. For example, if a potential therapeutic agent, say, agent A, causes a gene or group of genes of a characteristic or signature gene set, or even all of the genes of said gene set, to exhibit decreased expression, such as where a lower amount of mRNA is expressed from said gene(s), or less protein is produced from said mRNA, but a second potential agent, say, agent B, while modulating the activity of the same or related genes causes said expression to be reduced to half, such as where only half as much mRNA is transcribed or only half as much protein is translated from said mRNA as for agent A, then agent B is considered to have twice as much therapeutic potential as agent A.

Such modulation or change of activity as determined using the assays disclosed herein may include either an increase or a decrease in activity of said genes or gene sequences. Thus, where a gene is expressed in cancer cells but not in normal cells, or is up-regulated in cancer cells relative to normal cells, of the same organ or tissue type, an agent that down-regulates said gene or genes, or gene sequences, or prevents their expression entirely, is considered a potential antitumor agent within the present disclosure. Conversely, where an agent causes expression of a gene or genes, or gene sequences, expressed in normal cells but not in cancer cells, or where said agent up-regulates a gene or genes, or gene sequences, that are expressed in normal cells but not in cancer cells, or are up-regulated in normal cells but not in cancer cells, of the same organ or tissue type, said agent is considered to be a potential antitumor agent within the present disclosure.

The present invention also relates to a process that comprises a method for producing a product comprising identifying an agent according to one of the disclosed processes for identifying such an agent (such as the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

In accordance with the foregoing, the present invention further relates to a process for determining the cancerous status of a cell to be tested, comprising determining the level of expression in said cell of at least one gene that includes one of the nucleotide sequences selected from the sequences of SEQ ID NO: 1-8447, including sequences substantially identical to said sequences, or characteristic fragments thereof, or the complements of any of the foregoing and then comparing said expression to that of a cell known to be non-cancerous whereby the difference in said expression indicates that said cell to be tested is cancerous.

In specific embodiments of the present invention, said expression is determined for more than one of said genes, such as 2, 3, 4, 5, or more such genes, considered as a set, and even as many as a set of 10 such genes. A set of genes, for example, 5 such genes, may be found to be expressed at certain levels in cancer cells but are found to be expressed at lower levels (or not expressed at all) in non-cancerous, or normal, cells. Conversely, a set of, for example, 5 such genes may be found to be expressed in normal (i.e., non-cancerous) cells but expressed at lower levels (or not expressed at all) in cancer cells. Thus, by determining the set or pattern of genes expressed in cancer cells but expressed at lower levels (or not at all) in non-cancer, or vice versa, a method is achieved for diagnosing cancerous conditions wherein said genes are selected from those that include one of the sequences, or fragments of sequences, including complementary sequences, selected from SEQ ID NO: 1-8447. Using the methods disclosed herein, a diverse number of cancers can be readily detected using the methods of the present invention.

In accordance with the invention, although gene expression for a gene that includes as a portion thereof one of the nucleotide sequences of SEQ ID NO: 1-8447, is preferably determined by use of a probe that is a fragment of such nucleotide sequence, it is to be understood that the probe may be formed from a different portion of the gene. Thus, for each gene of the signature set of the present invention, the nucleotide sequence disclosed with respect to a specific sequence ID number may be only a portion of the nucleotide sequence that encodes expression of the gene. As a result, expression of the gene may be determined by use of a nucleotide probe that hybridizes to messenger RNA (mRNA) transcribed from a portion of the gene other than the specific nucleotide sequence disclosed with reference to a sequence ID number as recited herein.

The present invention further relates to a process for determining a cancer initiating, facilitating or suppressing gene comprising the steps of contacting a cell with a cancer modulating agent and determining a change in expression of a gene selected from the group consisting of the gene sequences of SEQ ID NO: 1-8447 and thereby identifying said gene as being a cancer initiating or facilitating gene.

Thus, some or all of the genes within the signature gene sets disclosed herein as SEQ ID NO: 1-8447 are found to play a direct role in the initiation or progression of cancer or even other diseases and disease processes. Because changes in expression of these genes (either up-regulation or down-regulation) are linked to the disease state (i.e. cancer), the change in expression may contribute to the initiation or progression of the disease. For example, if a gene that is up-regulated is an oncogene, or if a gene that is down-regulated is a tumor suppressor, such a gene provides for a means of screening for small molecule therapeutics beyond screens based upon expression output alone. For example, genes that display up-regulation in cancer and whose elevated expression contributes to initiation or progression of disease represent targets in screens for small molecules that inhibit or block their function. Examples include, but are not be limited to, kinase inhibition, cellular proliferation, substrate analogs that block the active site of protein targets, etc. Similarly, genes that display down-regulation in cancer and whose absence results in initiation or progression of disease are valuable therapeutics for gene therapy.

In accordance therewith, the present invention relates to a process for determining a cancer initiating or facilitating gene comprising contacting a cell expressing a test gene (one whose status as a cancer initiating or facilitating gene is to be determined) with an agent that decreases the expression of a gene corresponding to a polynucleotide having a sequence selected from the group consisting of SEQ ID NO: 1-8447, and detecting a decrease in expression of said test gene compared to when said agent is not present, thereby identifying said test gene as being a cancer initiating or facilitating gene. Such genes may, of course, be oncogenes and said decrease in expression may be due to a decrease in copy number of said gene in said cell or a cell derived from said cell, such as where copy number is reduced following cellular replication.

The present invention also relates to a process for determining a cancer suppressor gene comprising contacting a cell expressing a test gene (one whose status as a cancer suppressor gene is to be determined) with an agent that increases the expression of a gene that encodes an RNA at least 95% identical to an RNA encoded by (i.e., corresponds to) a polynucleotide having a sequence selected from the group consisting of SEQ ID NO: 1-8447 and detecting an increase in expression of said test gene compared to when said agent is not present, thereby identifying said test gene as being a cancer suppressor gene. The sequence identity may include identical sequences, as defined herein, and such a process includes embodiments wherein the increase in expression is due to an increase in copy number of the gene in said cell or a cell derived from said cell, such as by cellular replication. Such increase in expression may also include the induction of expression in a cell, especially a cancer cell, where such expression is not detectable in the absence of the agent.

It should be noted that there are a variety of different contexts in which genes have been evaluated as being involved in the cancerous process. Thus, some genes may be oncogenes and encode proteins that are directly involved in the cancerous process and thereby promote the occurrence of cancer in an animal. In addition, other genes may serve to suppress the cancerous state in a given cell or cell type and thereby work against a cancerous condition forming in an animal. Other genes may simply be involved either directly or indirectly in the cancerous process or condition and may serve in an ancillary capacity with respect to the cancerous state. All such types of genes are deemed with those to be determined in accordance with the invention as disclosed herein. Thus, the gene determined by said process of the invention may be an oncogene, or the gene determined by said process may be a cancer facilitating gene, the latter including a gene that directly or indirectly affects the cancerous process, either in the promotion of a cancerous condition or in facilitating the progress of cancerous growth or otherwise modulating the growth of cancer cells, either in vivo or ex vivo. In addition, the gene determined by said process may be a cancer suppressor gene, which gene works either directly or indirectly to suppress the initiation or progress of a cancerous condition. Such genes may work indirectly where their expression alters the activity of some other gene or gene expression product that is itself directly involved in initiating or facilitating the progress of a cancerous condition. For example, a gene that encodes a polypeptide, either wild or mutant in type, which polypeptide acts to suppress of tumor suppressor gene, or its expression product, will thereby act indirectly to promote tumor growth.

In accordance with the foregoing, the process of the present invention includes cancer modulating agents that are themselves either polypeptides, or small chemical entities, that affect the cancerous process, including initiation, suppression or facilitation of tumor growth, either in vivo or ex vivo. Said cancer modulating agent may have the effect of increasing gene expression or said cancer modulating agent may have the effect of decreasing gene expression as such terms have been described herein.

In keeping with the present disclosure, the present invention also relates to a process for treating cancer comprising contacting a cancerous cell with an agent having activity against an expression product encoded by a gene sequence selected from the group consisting of SEQ ID NO: 1-8447. More specifically, the present invention relates to a process for treating cancer comprising contacting a cancerous cell with an agent having activity against an expression product encoded by a gene sequence selected from the group consisting of SEQ ID NO: 1-8447. Such a process includes an embodiment wherein the cancerous cell is contacted in vivo. Such treatment includes treatment of a patient, such as a human being. The agent may include an antibody that reacts with a polypeptide encoded by such a gene.

Thus, some or all of the genes within these signature gene sets represent individual targets for therapeutic intervention, based at least in part on their pattern(s) of expression. For example, genes within the signature gene sets that encode cell surface molecules and are up-regulated in cancer as compared to normal cells. The proteins encoded by such genes, due to their elevated expression in cancer cells, represent highly useful therapeutic targets for “targeted therapies” utilizing such affinity structures as, for example, antibodies coupled to some cytotoxic agent. In such methodology, it is advantageous that nothing need be known about the endogenous ligands or binding partners for such cell surface molecules. Rather, an antibody or equivalent molecule that can specifically recognize the cell surface molecule (which could include an artificial peptide, a surrogate ligand, and the like) that is coupled to some agent that can induce cell death or a block in cell cycling offers therapeutic promise against these proteins. Thus, such approaches include the use of so-called suicide “bullets” against intracellular proteins

The process of the present invention includes embodiments of the above-recited process wherein said cancer cell is contacted in vivo as well as ex vivo, preferably wherein said agent comprises a portion, or is part of an overall molecular structure, having affinity for said expression product. In one such embodiment, said portion having affinity for said expression product is an antibody, especially where said expression product is a polypeptide or oligopeptide or comprises an oligopeptide portion, or comprises a polypeptide.

Such an agent can therefore be a single molecular structure, comprising both affinity portion and anti-cancer activity portions, wherein said portions are derived from separate molecules, or molecular structures, possessing such activity when separated and wherein such agent has been formed by combining said portions into one larger molecular structure, such as where said portions are combined into the form of an adduct. Said anti-cancer and affinity portions may be joined covalently, such as in the form of a single polypeptide, or polypeptide-like, structure or may be joined non-covalently, such as by hydrophobic or electrostatic interactions, such structures having been formed by means well known in the chemical arts. Alternatively, the anti-cancer and affinity portions may be formed from separate domains of a single molecule that exhibits, as part of the same chemical structure, more than one activity wherein one of the activities is against cancer cells, or tumor formation or growth, and the other activity is affinity for an expression product produced by expression of genes related to the cancerous process or condition.

In one embodiment of the present invention, a chemical agent, such as a protein or other polypeptide, is joined to an agent, such as an antibody, having affinity for an expression product of a cancerous cell, such as a polypeptide or protein encoded by a gene related to the cancerous process, especially a gene sequence corresponding to one selected from the group consisting of the sequences of SEQ ID NO: 1-8447. In a specific embodiment, said expression product is a cell surface receptor, such as a protein or glycoprotein or lipoprotein, present on the surface of a cancer cell, such as where it is part of the plasma membrane of said cancer cell, and acts as a therapeutic target for the affinity portion of said anticancer agent and where, after binding of the affinity portion of such agent to the expression product, the anti-cancer portion of said agent acts against said expression product so as to neutralize its effects in initiating, facilitating or promoting tumor formation and/or growth. In a separate embodiment of the present invention, binding of the agent to said expression product may, without more, have the effect of deterring cancer promotion, facilitation or growth, especially where the presence of said expression product is related, either intimately or only in an ancillary manner, to the development and growth of a tumor. Thus, where the presence of said expression product is essential to tumor initiation and/or growth, binding of said agent to said expression product will have the effect of negating said tumor promoting activity. In one such embodiment, said agent is an apoptosis-inducing agent that induces cell suicide, thereby killing the cancer cell and halting tumor growth.

In alternative embodiments of the foregoing, the present invention relates to a process for treating a cancerous condition in an animal afflicted therewith comprising administering to said animal a therapeutically effective amount of an agent first identified as having anti-neoplastic activity using an assay process as disclosed herein according to the present invention, such as a cancer-related gene modulator as identified according to the processes of the invention. Such processes also include the ability to protect against development of a cancerous state by using agents identified by the assay processes of the invention. Thus, the present invention specifically contemplates a process for protecting an animal against cancer comprising administering to an animal at risk of developing cancer a therapeutically effective amount of an agent first identified as having anti-neoplastic activity using one or more of the assay processes disclosed herein for identifying such agents.

The processes of the present invention take advantage of the correlation of changes in mRNA expression profiles of these signature gene sets with potential (depending on the form of cancer) changes in DNA copy number of the chromosomal regions wherein these genes are located. Of course, the precise nature of the change in mRNA expression (e.g. a signature set of genes that are up-regulated at the transcriptional level) may also indicate a change in the DNA copy number for the genomic regions in which these genes are located (e.g. an amplification of the genomic DNA region that contains the involved gene or genes).

Many cancers contain chromosomal rearrangements, which typically represent translocations, amplifications, or deletions of specific regions of genomic DNA. A recurrent chromosomal rearrangement that is associated with a specific stage and type of cancer always affects a gene (or possibly genes) that play a direct and critical role in the initiation or progression of the disease. Many of the known oncogenes or tumor suppressor genes that play direct roles in cancer have either been initially identified based upon their positional cloning from a recurrent chromosomal rearrangement or have been demonstrated to fall within a rearrangement subsequent to their cloning by other methods. In all cases, such genes display amplification at both the level of DNA copy number and at the level of transcriptional expression at the mRNA level.

At least some of the genes that are contained within signature gene sets disclosed herein (SEQ ID NO: 1-8447) display changes in their mRNA expression profiles (depending on the precise reading frame involved) within cancer samples due, in part, to changes in their DNA copy number as a result of specific chromosomal rearrangements in those cancer cells. The utilities that follow from this are (i) that the genes contained within these signature gene sets offer a time saving shortcut to the identification of novel chromosomal rearrangements, amplifications, or deletions that are associated with cancer, and/or (ii) represent key genes affected by such chromosomal rearrangements, amplifications, or deletions and, therefore, play a key role in the initiation or progression of the disease. Genes within the signature sets that identify changes in the DNA copy number (based upon their changes in expression at the mRNA level) afford an entry point into other forms of diagnostic assay for the initiation, staging, or progression of cancer to be conducted in tissue samples at the DNA level (e.g. if gene X identifies a novel chromosomal amplification associated with cancer, then that specific chromosomal region defined by gene X would serve as the basis for a diagnostic assay for cancer, where genomic DNA is extracted from tissue samples and evaluated for the presence of the specific amplification), and also the rapid positional cloning of genes that play vital and direct roles in the initiation or progression of cancer.

In one embodiment of the present invention, said change in expression may be determined by determining a change in gene copy number, wherein said change in copy number is an increase in copy number or wherein said change in copy number is a decrease in copy number. For example, copy number of a sequence expressed, or over-expressed, in a cancerous cell may be decreased due to the presence of an anti-neoplastic agent as identified according to the assays procedures of the present invention.

A change in gene copy number may be determined by determining a change in expression of messenger RNA encoded by a particular gene sequence, especially where said sequence is one selected from the group consisting of the sequences of SEQ ID NO: 1-8447, some being expressed in cancer cells but not expressed at detectable levels in normal cells and others being expressed in normal cells but not at detectable levels in cancer cells. Also in accordance with the present invention, said gene may be a cancer initiating gene, a cancer facilitating gene, or a cancer suppressing gene. In carrying out the methods of the present invention, a cancer facilitating gene is a gene that, while not directly initiating or suppressing tumor formation or growth, said gene acts, such as through the actions of its expression product, to direct, enhance, or otherwise facilitate the progress of the cancerous condition, including where such gene acts against genes, or gene expression products, that would otherwise have the effect of decreasing tumor formation and/or growth.

The present invention also relates to a process for treating cancer comprising inserting into a cancerous cell a gene construct comprising an anti-cancer gene operably linked to a promoter or enhancer element such that expression of said anti-cancer gene causes suppression of said cancer and wherein said promoter or enhancer element is a promoter or enhancer element modulating a gene, or genes, corresponding to a sequence, or sequences, selected from the group consisting of the sequences of SEQ ID NO: 1-8447.

The signature sets or signature gene sets disclosed herein are useful in identifying genetic regulatory elements within the promoters of the genes contained within the signature sets that are specific to normal tissue and/or the corresponding cancer. Each signature set is a collection of genes that share a gross common pattern of transcriptional regulation in cancer vs. normal (e.g. a signature set of genes that are transcriptionally up-regulated in cancer).

In one such embodiment, analyzing and comparing the DNA sequences of the promoter regions of all the genes contained within the signature set serves to identify conserved stretches or motifs of sequences within subsets of genes that represent cis-acting elements that specifically drive a form of gene expression (e.g. increased transcriptional expression in cancer). The identification of such cis-acting regulatory elements is then available for use in driving the cancer-specific expression of suicide genes or toxins via genetic therapy using technology already well known in the art.

In separate embodiments, said anti-cancer gene is a cancer suppressor gene or encodes a polypeptide having anticancer activity, especially where said polypeptide has apoptotic activity.

In additional embodiments, such insertion of the gene construct into a cancerous cell is accomplished in vivo, for example using a viral or plasmid vector. Such methods can also be applied to in vitro uses. The methods of the present invention are readily applicable to different forms of gene therapy, either where cells are genetically modified ex vivo and then administered to a host or where the gene modification is conducted in vivo using any of a number of suitable methods involving vectors especially suitable to such therapies, such as the use of special viral vectors, including adeno-associated viruses and adenoviruses, as well as retroviruses and specially constructed plasmids to accomplish such therapies. The use of these and other vectors is well known to those skilled in the art and need not be described further.

The present method also relates to a process for determining functionally related genes comprising contacting one or more gene sequences selected from the group consisting of the sequences of SEQ ID NO: 1-8447 with an agent that modulates expression of more than one gene in such group and thereby determining a subset of genes of said group.

In accordance with the present invention, said functionally related genes are genes modulating the same metabolic pathway or said genes are genes encoding functionally related polypeptides. In one such embodiment, said genes are genes whose expression is modulated by the same transcriptional activator or enhancer sequence, especially where said transcriptional activator or enhancer increases, or otherwise modulates, the activity of a gene sequence selected from the group consisting of SEQ ID NO: 1-8447. In specific embodiments, the sequences may be subsets of these.

Thus, the signature gene set disclosed herein also find use as the basis for small molecule assays for therapeutics based upon changes in expression profile. In one such embodiment, small molecule screens serve to identify changes in expression of genes within a signature set and thereby provide a tool for the identification of specific functional pathways and a means of assigning defined functions to novel genes.

In accordance with the foregoing, monitoring the transcriptional expression of the genes contained within the signature sets disclosed herein forms the basis of an assay for small molecule therapeutics. For example, in situations where a signature set of genes that are transcriptionally up-regulated in cancer cells compared to normal cells, such screens facilitate the identification of small molecules that down-regulate the expression of the genes of the signature set within cancer cells. While such therapeutics make a cancer cell “look” more normal, based upon the expression of the genes within the signature set, what actually happens when such screens are put into practice is that all genes within the signature sets do not respond identically to each small molecule within a chemical compound library. If an average signature set contains 200 different genes, for example, and the expression of all 200 genes is monitored in response to a library of some 50,000 chemical compounds, and subsets of genes within the signature set consistently change their patterns of expression in response to particular chemicals (e.g., 10 of the genes always change expression in a coordinated way, such as down-regulation of one gene within the group of 10) then it always causes the down-regulation of the other 9 specific genes as well.

Such subsets or subgroups of genes within each signature set that change their expression in a coordinated way in response to chemical compounds represent genes that are located within a common metabolic, signaling, physiological, or functional pathway so that by analyzing and identifying such subsets one can (a) assign known genes and novel genes to specific pathways and (b) identify specific functions and functional roles for novel genes that are grouped into pathways with genes for which their functions are already characterized or described. For example, one might identify a subgroup of 10 genes within a signature set (5 known genes & 5 novel genes) that change expression in a coordinated fashion and for which the 5 known genes are involved in apoptosis thereby implicating the other 5 novel genes as playing a role in apoptotic cellular processes. Therefore, the processes disclosed according to the present invention at once provide a novel means of assigning function to genes, i.e. a novel method of functional genomics, and a means for identifying chemical compounds that have potential therapeutic effects on specific cellular pathways. Such chemical compounds may have therapeutic relevance to a variety of diseases outside of cancer as well, in cases where such diseases are known or are demonstrated to involve the specific cellular pathway that is affected.

It should be cautioned that, in carrying out the procedures of the present invention as disclosed herein, any reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make substitutions that will optimally serve their purposes in using the methods and procedures disclosed herein.

The present invention will now be further described by way of the following non-limiting example but it should be kept clearly in mind that other and different embodiments of the methods disclosed according to the present invention will no doubt suggest themselves to those of skill in the relevant art.

### EXAMPLE

SW480 cells are grown to a density of 10⁵ cells/cm² in Leibovitz's L-15 medium supplemented with 2 mM L-glutamine (90%) and 10% fetal bovine serum. The cells are collected after treatment with 0.25% trypsin, 0.02% EDTA at 37° C. for 2 to 5 minutes. The trypsinized cells are then diluted with 30 ml growth medium and plated at a density of 50,000 cells per well in a 96 well plate (200 μl/well). The following day, cells are treated with either compound buffer alone, or compound buffer containing a chemical agent to be tested, for 24 hours. The medium is then removed, the cells lysed and the RNA recovered using the RNAeasy reagents and protocol obtained from Qiagen. RNA is quantitated and 10 ng of sample in 1 μl are added to 24 μl of Taqman reaction mix containing 1×PCR buffer, RNAsin, reverse transcriptase, nucleoside triphosphates, amplitaq gold, Tween 20, glycerol, bovine serum albumin (BSA) and specific PCR primers and probes for a reference gene (18S RNA) and a test gene (Gene X). Reverse transcription is then carried out at 48° C. for 30 minutes. The sample is then applied to a Perkin Elmer 7700 sequence detector and heat denatured for 10 minutes at 95° C. Amplification is performed through 40 cycles using 15 seconds annealing at 60° C. followed by a 60 second extension at 72° C. and 30 second denaturation at 95° C. Data files are then captured and the data analyzed with the appropriate baseline windows and thresholds.

The quantitative difference between the target and reference genes is then calculated and a relative expression value determined for all of the samples used. This procedure is then repeated for each of the target genes in a given signature, or characteristic, set and the relative expression ratios for each pair of genes is determined (i.e., a ratio of expression is determined for each target gene versus each of the other genes for which expression is measured, where each gene's absolute expression is determined relative to the reference gene for each compound, or chemical agent, to be screened). The samples are then scored and ranked according to the degree of alteration of the expression profile in the treated samples relative to the control. The overall expression of the set of genes relative to the controls, as modulated by one chemical agent relative to another, is also ascertained. Chemical agents having the most effect on a given gene, or set of genes, are considered the most anti-neoplastic.

In carrying out the methods of the invention, it is to be expected that not all cells of a given sample of suspected cancerous cells will express all, or even most, of these genes but that a substantial expression thereof in a substantial number of such cells is sufficient to warrant a determination of a cancerous, or potentially cancerous, condition. The sequences disclosed herein are represented by SEQ ID NO: 1 to 8447 although different genes more or less relevant to different organs and tissues and some may be up-regulated in cancer and not normal cells while others are up-regulated in normal cells but not cancerous cells. The sequences presented herein may be genomic, synthetic or cDNA sequences and may also be represented as RNA sequences. The sequences of the sequence listing herein are mostly cDNA sequences but can be used to locate genomic sequences.

What is claimed is:

1. A process for screening a plurality of chemical compounds for anti-neoplastic activity identifying a gene modulating agent comprising:
(a) contacting a compound with one or more cells expressing a gene encoding an RNA also encoded by a nucleotide sequence selected from SEQ ID NO: 1-8447, and
(b) determining a change in expression of at least one said gene due to said contacting,
wherein an increase in the expression of the determined genes whose expression is elevated in a non-cancerous cell over that in a cancerous cell of the same tissue type and a decrease in the expression of the determined genes whose expression is increased in a cancerous cell over that in a non-cancerous cell of the same tissue type
is indicative of anti-neoplastic activity.

2. A process for determining the cancerous status of a test cell, comprising determining expression in said test cell of at least one gene that includes one of the nucleotide sequences selected from the sequences of SEQ ID NOS: 1-8447, or a nucleotide sequence that is at least 95% identical thereto, and then comparing said expression to expression of said at least one gene in at least one cell known to be non-cancerous whereby a difference in said expression indicates that said cell is cancerous.

3. A process for treating cancer comprising contacting a cancerous cell with an agent having activity against an expression product encoded by a gene sequence selected from the group consisting of SEQ ID NO: 1-8447.

4. The process of claim 3 wherein said cancer is selected from the group consisting of colon cancer, lung cancer, ovarian cancer, pancreatic cancer, thyroid cancer, stomach cancer, prostate cancer, kidney cancer, esophageal cancer and breast cancer.

5. A method for identifying a compound as an anti-neoplastic agent, comprising:
(a) contacting a test compound with a gene product encoded by a polynucleotide of SEQ ID NO: 1-8447,
(b) determining a change in a biological activity of said gene product due to said contacting,
wherein a change in activity identifies said test compound as an agent having antineoplastic activity.

6. The method of claim 5 wherein said gene product is a polypeptide.

7. The method of claim 6 wherein said biological activity is an enzyme activity.

8. The method of claim 6 wherein said test compound is a substrate analog that blocks the active site of said polypeptide.

9. The method of claim 6 wherein said polypeptide is a cell-surface polypeptide.

10. The method of claim 6 wherein said polypeptide is a receptor and the test compound causes a change in biological activity by blocking binding of a ligand to said receptor.

11. An antibody that binds to a polypeptide comprising an amino acid sequence encoded by a polynucleotide having a sequence of SEQ ID NO: 1-8447.

12. An immunoconjugate comprising an affinity portion and an anti-cancer portion wherein said affinity portion is an antibody of claim 11.

13. The immunoconjugate of claim 12 wherein said anti-cancer portion is a cytotoxic agent.

14. The immunoconjugate of claim 13 wherein said cytotoxic agent is an agent that induces cell death.

15. The immunoconjugate of claim 13 wherein said cytotoxic agent is an agent that blocks the cell cycle.

16. The immunoconjugate of claim 13 wherein said cytotoxic agent is an apoptosis-inducing agent

17. A process for treating cancer comprising contacting a cancerous cell in vivo with an agent having activity against an expression product encoded by a gene sequence selected from the group consisting of polynucleotide sequences of SEQ ID NO: 1-8447.

18. The process of claim 17 wherein said agent is an antibody.

19. The process of claim 17 wherein said agent is an immunoconjugate of claim 31.

20. The process of claim 17 wherein said agent is an immunoconjugate of claim 32.
